(19) United States
(12) Patent Application Publication
(10) Pub. No.: US 2019/0192843 A1
(43) Pub. Date: Jun. 27, 2019

(54) COUPLER FOR CLEANING A FEEDING TUBE HUB

(71) Applicant: NEOMED, INC., Woodstock, GA (US)

(72) Inventors: Benjamin M. DAVIS, Woodstock, GA (US); David A. DOORNBOS, Woodstock, GA (US); Aaron N. INGRAM, Canton, GA (US); Rommell Gregory NEU, Mableton, GA (US)

(73) Assignee: NEOMED, INC., Woodstock, GA (US)

(21) Appl. No.: 16/288,377

(22) Filed: Feb. 28, 2019

Related U.S. Application Data
(63) Continuation-in-part of application No. 15/009,073, filed on Jan. 28, 2016, now Pat. No. 9,931,176.
Application No. 15/009,073 is a continuation-in-part of application No. 16/223,917, filed on Dec. 18, 2018.
Application No. 16/223,917 is a continuation-in-part of application No. 16/154,479, filed on Oct. 8, 2018.
(60) Provisional application No. 62/636,924, filed on Mar. 1, 2018.
(60) Provisional application No. 62/108,824, filed on Jan. 28, 2015.

(51) Int. Cl. A61M 39/16; A61J 15/00; A61M 25/00

(52) U.S. Cl. CPC A61M 39/16; A61J 15/0026; A61M 2209/10; A61M 2025/0019; A61M 25/0097

(57) ABSTRACT

A plug for plugging a feeding tube hub that has a male coupling defining a lumen extending therethrough, and an outer collar and an annular space defined therebetween. The plug includes a base configured for releasably covering at least a portion of an annular space defined in an outer collar of a feeding tube hub, and a plug insert extending from the base. The plug insert is configured for plugging a lumen of a feeding tube hub along a connection axis. At least one passageway extends through the base to provide access to the annular space defined in the outer collar of the feeding tube hub when the plug insert is plugging the lumen of the feeding tube hub.

### CROSS-REFERENCE TO RELATED APPLICATION

This application claims the priority benefit of U.S. Provisional Patent Application Ser. No. 62/636,924 filed Mar. 1, 2018, is a Continuation-in-Part application of U.S. Non-provisional patent application Ser. No. 15/009,073 filed Jan. 28, 2016, which claims the priority benefit of U.S. Provisional Patent Application Ser. No. 62/108,824 filed Jan. 28, 2015, is a Continuation-in-Part application of U.S. Non-provisional patent application Ser. No. 16/223,917 filed Dec. 18, 2018, and is a Continuation-in-Part application of U.S. Non-provisional patent application Ser. No. 16/154,479 filed Oct. 8, 2018, the entireties of which are hereby incorporated by reference for all purposes.

### TECHNICAL FIELD

The present invention relates generally to the field of medical devices, and more particularly to cleaning devices and methods for use with fluid transfer connectors such as coupling connectors for enteral fluid containers.

### BACKGROUND

Neonates and other healthcare patients are often administered fluids such as medications, nutritional fluids and supplements via enteral fluid delivery, commonly utilizing delivery systems including fluid containers, syringes, feeding tubes and other components. These components are often interconnected by connectors or couplings such as Luer connectors, or the more recently developed ENFit connector (ISO Standard 80369-3).

In some embodiments, these enteral connectors or couplings may include outer housing geometries with recesses or areas that could retain small quantities of unused feeding fluids that might allow for bacteria colonization or contain other potential contaminants. U.S. patent application Ser. No. 14/844,956, which is incorporated herein by reference, discloses a vented male ENFit enteral coupling or connector having a housing structure with drainage passages or vents to eliminate or reduce the likelihood of retaining feeding liquids or other contaminants in the outer housing. Cleaning tools as disclosed in U.S. Non-provisional patent application Ser. No. 15/009,073 filed Jan. 28, 2016, U.S. Provisional Patent Application Ser. No. 62/108,824 filed Jan. 28, 2015, and U.S. Provisional Patent Application Ser. No. 62/599,912 filed Dec. 18, 2017, the entireties of which are hereby incorporated by reference for all purposes, have been developed. In some cases, multiple manufacturing processes and molds are required to manufacture the cleaning tools which lead to higher costs. Improvements in the field are desirable, and it is to the provision of low-cost cleaning tools and methods thereof that the present invention is primarily directed.

### SUMMARY

In example embodiments, the present invention provides a cleaning tool and methods thereof for cleaning a feeding tube hub.

In one aspect, the present invention relates to feeding tube hub plug for plugging a feeding tube hub. The feeding tube hub includes a male coupling defining a lumen extending therethrough, an outer collar and an annular space defined therebetween, the feeding tube hub plug includes a central plug member for plugging a lumen of a feeding tube hub and a body extending from the central plug member. In example embodiments, at least one opening or cutout is defined in the body such that the annular space is accessible without unplugging the central plug member from the lumen of the feeding tube hub.

In another aspect, the invention relates to a cleaning tool for cleaning a feeding tube hub including a brush member having a brush end and a grasping portion connected to the brush end, the brush member including a plurality of bristles. In example embodiments, the feeding tube hub includes a plug having one or more openings for receiving at least a portion of the cleaning tool to clean the feeding tube hub.

In still another aspect, the invention relates to a cleaning system for enteral feeding including a feeding tube hub plug and a cleaning tool. In example embodiments, the feeding tube hub plug includes a central plug member, a body extending from the central plug member, and at least one opening or cutout defined in the body. The cleaning tool includes an elongate member having a brush end and a grasping portion. The brush end includes a plurality of bristles extending therefrom. In example embodiments, at least a portion of the brush end is configured for passing through the at least one opening or cutout to access an annular space portion of the feeding tube hub.

In yet another aspect, the invention relates to a male to female coupling for attachment between a connector and a syringe. In example embodiments, the coupling is configured for coupling engagement between a male ENFit formatted coupling of a feeding tube connector and a female ENFit formatted coupling of a syringe.

In yet another aspect, the invention relates to method of cleaning a feeding tube hub including providing a feeding tube hub, the feeding tube hub being in communication with a lumen of a feeding tube and having a male coupling defining a lumen extending therethrough, an outer collar and an annular space defined therebetween; providing a cleaning tool; providing a feeding tube hub plug, the feeding tube hub plug having a plug member for sealing engagement with the lumen of the male coupling, a body extending from the plug member, and at least one opening or cutout defined in the body; attaching the plug member with the feeding tube hub to seal the lumen of the male coupling; inserting at least a portion of the cleaning tool within the at least one opening or cutout of the feeding tube plug hub such that at least a portion of the brush end is present within the annular space of the feeding tube hub; rotating the feeding tube hub plug relative to the feeding tube hub while the cleaning tool remains at least partially present within the annular space; and removing the cleaning tool from the at least one opening or cutout of the feeding tube hub plug.

In yet another aspect, the invention relates to a plug for a feeding tube connector including a body, a central plug member, a syringe tip coupling and one or more channels extending through the body. In example embodiments, the central plug member extends from a first side of the body for plugging a lumen of a feeding tube hub. In example embodiments, the syringe tip coupling extends from a second side of the body and is configured for compatible engagement with a syringe tip of a syringe. In example embodiments, the one or more channels extending through the body are configured such that a cleaning fluid introduced within the syringe tip coupling flows through the body and within the annular space to clean the same.

In a further aspect, the present disclosure relates to a plug for plugging a feeding tube hub that has a male coupling defining a lumen extending therethrough, and an outer collar and an annular space defined therebetween. The plug includes a base configured for releasably covering at least a portion of an annular space defined in an outer collar of a feeding tube hub, and a plug insert extending from the base. The plug insert is configured for plugging a lumen of a feeding tube hub along a connection axis. At least one passageway extends through the base to provide access to the annular space defined in the outer collar of the feeding tube hub when the plug insert is plugging the lumen of the feeding tube hub.

In an additional aspect, the present disclosure relates to a coupler for cleaning a feeding tube hub comprising a male coupling defining a lumen extending therethrough, an outer collar and an annular space defined therebetween, the coupler comprising: a base configured for releasably covering at least a portion of an annular space defined in an outer collar of a feeding tube hub; a seal configured for removably sealing a lumen of a feeding tube hub; and a fluid conduit configured for receiving a volume of fluid from a fluid source and delivering the volume of fluid to the annular space in the outer collar of the feeding tube hub when the seal is sealing the feeding tube lumen.

In another aspect, the present disclosure relates to a coupling system comprising: a fluid delivery female ISO 80369-3 formatted coupling; a hub male ISO 80369-3 formatted coupling being separate from the fluid delivery female ISO-80369-3 formatted coupling; and a coupler comprising a female ISO 80369-3 formatted coupling and a male ISO 80369-3 formatted coupling, the coupler being separate from the fluid delivery female ISO 80369-3 formatted coupling and the hub male ISO 80369-3 formatted coupling, the coupler female ISO 80369-3 formatted coupling being removably coupled to the hub male ISO 80369-3 formatted coupling, and the coupler male ISO 80369-3 formatted coupling being removably coupled to the fluid delivery female ISO 80369-3 formatted coupling.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of example embodiments are explanatory of example embodiments of the invention, and are not restrictive of the invention, as claimed.

### BRIEF DESCRIPTION OF THE DRAWINGS

FIG. 1 is a perspective view of a schematic diagram of a feeding tube hub plug plugged into a feeding tube hub with a cleaning tool inserted therein, according to an example embodiment of the present disclosure.

FIG. 2 is an exploded view of the feeding tube hub, feeding tube hub plug and cleaning tool shown in FIG. 1.

FIG. 3 is an isolated perspective view of the feeding tube hub plug shown in FIGS. 1-2.

FIG. 4 is a side view of the feeding tube hub plug shown in FIG. 3.

FIG. 5 is a top view of the feeding tube hub plug shown in FIG. 3.

FIGS. 6-13 are a plurality of top views of feeding tube hub plug schematic diagrams, according to additional example embodiments of the present disclosure, which can cooperatively function with the feeding tube hub and the cleaning tool shown in FIGS. 1-2.

FIG. 14 is a top perspective view of a schematic diagram of a feeding tube hub plug with a syringe tip connector, according to another example embodiment of the present disclosure.

FIG. 15 is a bottom perspective view of the feeding tube hub plug shown in FIG. 14.

FIG. 16 is a perspective view of a schematic diagram of a system including a feeding tube hub, a syringe and a male to female connector, according to another example embodiment of the present disclosure.

FIG. 17 is an exploded view of the system shown in FIG. 16.

FIG. 18 is an isolated front perspective view of the male to female connector shown in FIGS. 16-17.

FIG. 19 is a rear perspective view of the male to female connector shown in FIG. 18.

FIG. 20 is a perspective view of a schematic diagram of a male to female connector with clip connectors, according to another example embodiment of the present disclosure.

FIG. 21 is a front perspective view of a schematic diagram of a male to female connector and feeding tube hub plug, according to another example embodiment of the present disclosure.

FIG. 22 is a rear perspective view of the male to female connector and feeding tube hub plug shown in FIG. 21.

FIG. 23 is a front view of the male to female connector and feeding tube hub plug shown in FIGS. 21-22.

FIG. 24 is a rear view of the male to female connector and feeding tube hub plug shown in FIGS. 21-22.

### DETAILED DESCRIPTION OF EXAMPLE EMBODIMENTS

The present invention may be understood more readily by reference to the following detailed description of example embodiments taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Any and all patents and other publications identified in this specification are incorporated by reference as though fully set forth herein.

Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

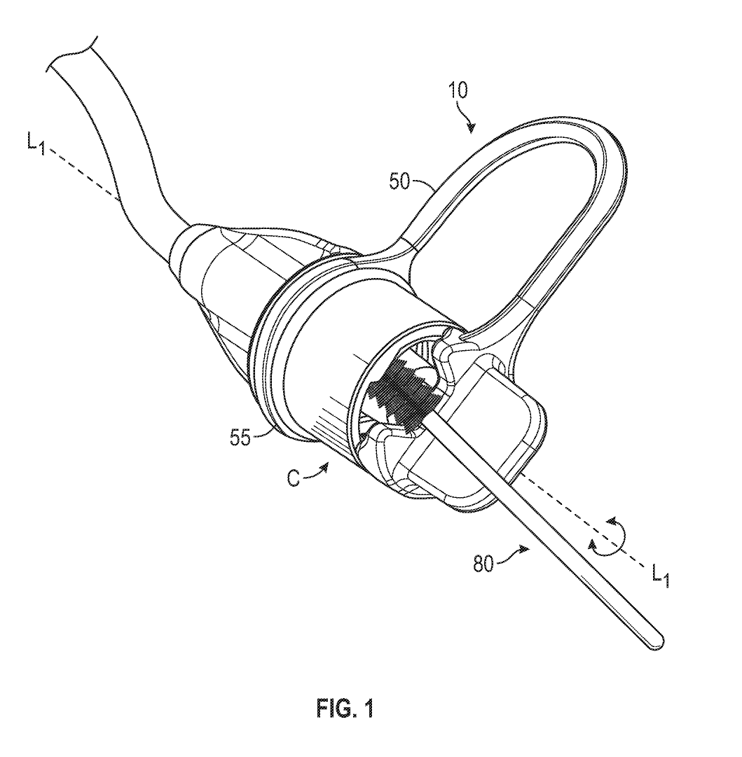

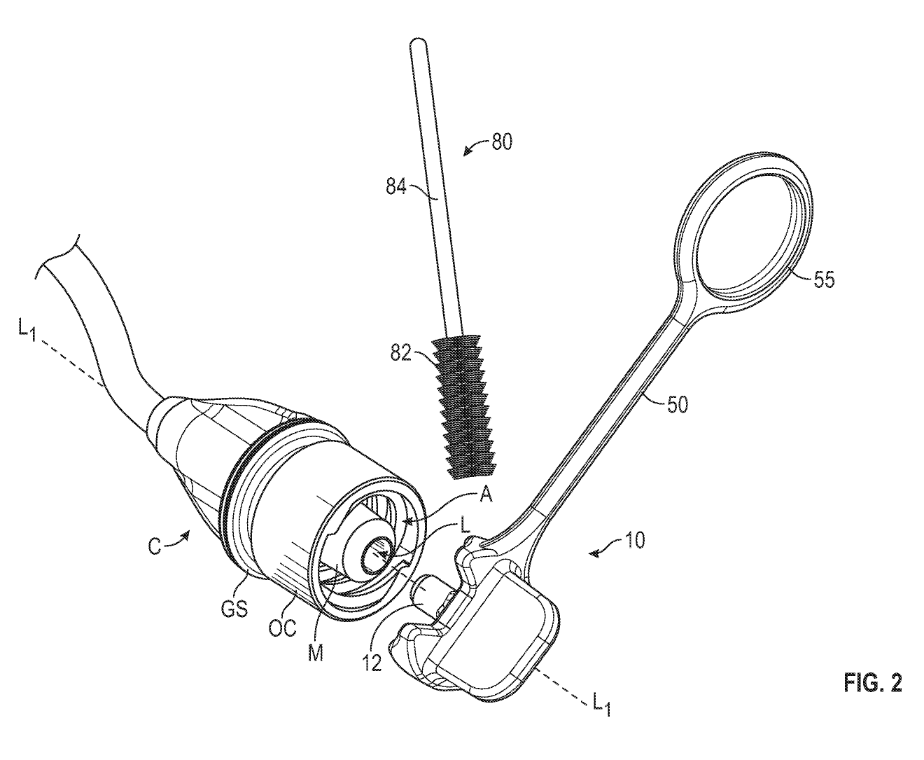

With reference now to the drawing figures, wherein like reference numbers represent corresponding parts throughout the several views, FIGS. 1-2 show a feeding tube hub plug 10 for sealing a feeding tube hub or connector C along a connection axis L₁ according to example embodiments of the present invention. In example embodiments, the plug 10 comprises a plug member 12 or insert for sealing or plugging a lumen L of a coupling or connector M of the feeding tube connector C. In example embodiments, the feeding tube connector C is substantially related to U.S. patent application Ser. No. 14/844,956, which is incorporated herein by reference herein, and discloses a vented male ISO 80369-3 formatted ENFit enteral coupling or connector having a housing structure with drainage passages or vents to eliminate or reduce the likelihood of retaining feeding liquids or other contaminants in the outer housing. The plug 10 can have a tether 50 with an end ring 55 or fastener which secures to a grooved surface GS or recess on the connector C. The grooved surface GS can be a channel extending circumferentially around the connector C. The tether 50 can be flexible and can form a shape like a handle, as shown in FIG. 1, to allow a user to grasp and rotate the plug 10 about the connection axis L₁ with respect to the connector C.

According to example embodiments, an annular space A is defined between the connector M and a threaded inner surface of an outer collar OC, which may retain small quantities of unused feeding fluids that might allow for bacteria colonization or contain other potential contaminants. Accordingly, preferably the plug 10 as described herein allows for effectively cleaning the annular space A of the connector C while the lumen L of the connector M remains sealed by the plug member 12. One or more openings (described further below) are defined in at least a portion of the plug 10 to provide access to the annular space A such that a brush or other cleaning member 80 can be inserted through one or more openings and within the annular space A to clean the same.

In example embodiments, the cleaning tool 80 comprises a generally elongate brush member comprising a brush end 82 and a grasping end 84. The brush end 82 comprises a plurality of flexible (e.g., nylon) bristles extending radially from a rigid (e.g., stainless steel) post, and the grasping portion 84 comprises a rigid (e.g., plastic) shaft for controlling the actions of the brush end. Optionally, the cleaning tool 80 can be formed from other plastics, metals, natural materials, synthetic materials, and/or combinations thereof. In example embodiments, the brush end 82 generally extends along a linear path and is co-aligned with the grasping portion 84. In other example embodiments, the brush end 82 can extend along a linear path that is angled relative to the grasping portion 84, or for example, one or more portions of the brush end can extend along a non-linear path.

According to some example embodiments, the cleaning tool 80 can be wetted or coated with a liquid or cleaning solution or fluid prior to being inserted through the one or more openings. Optionally, a cleaning fluid can be provided within the annular space A prior to inserting the cleaning tool 80 therein to clean. According to one example embodiment, the cleaning fluid is contained within a container or other reservoir that is capable of discharging at least one fluid through the one or more openings 30 and within the annular space A. According to some example embodiments, a coupling or other connector can be provided on a portion of the base 20 (and in communication with the annular space A) such that a syringe or other container or dispensing means can be connected to the coupling for discharging the cleaning fluid within the annular space. Thereafter, the brush end 82 can be inserted through the one or more openings and cleaning can begin. According to additional example embodiments, the plug can further comprise a coupling for permitting a syringe or other device containing cleaning fluid to connect thereto for applying the cleaning fluid within the annular space.

After the feeding tube hub C is used for feeding a patient, the plug 10 is reconnected with the hub C such that the plug member 12 seals with the lumen L of the hub C. The brush end 82 of the cleaning tool 80 is then inserted within the opening 30 such that the brush end 82 generally occupies at least a portion of the annular space A. Then, with the brush end 82 occupying at least a portion of the annular space A and with the plug member 12 still being sealed with the lumen L, the entirety of the plug 10 and cleaning tool 80 are rotated about rotation about the connection axis L₁ with respect to the hub C (e.g., 360° of according to some example embodiments) to sweep any debris to the bottom of the hub. According to some example embodiments, after rotation of the plug 10 and cleaning tool 80, the cleaning tool 80 is further inserted to pass through the annular space A and extend out of a bottom portion of the hub C, for example, wherein at least the brush end 82 extends through a vent feature that is generally provided at a bottom portion of the hub C. Thereafter, with the plug 10 remaining engaged with the hub C, the cleaning cool 80 is removed from the opening and discarded. According to some example embodiments, the cleaning tool 80 can be configured for multiple uses or cleanings, for example, wherein the entirety of the cleaning tool 80 or just a portion thereof can be cleaned and stored for additional cleanings.

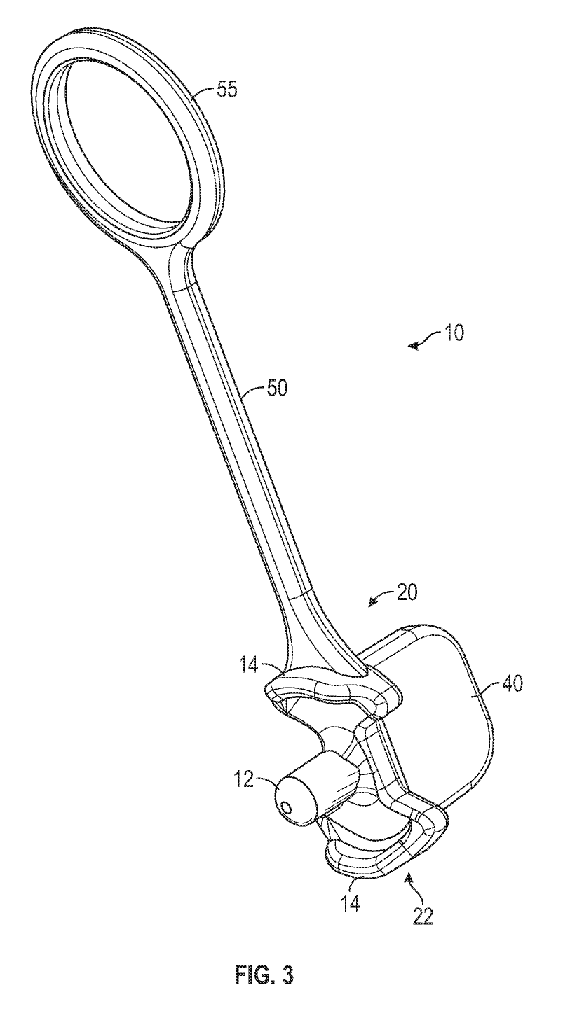

The plug 10 can preferably be configured as desired to permit accessibility of the cleaning tool 80 within the annular space A and with the plug member 12 sealing the lumen L of the connector M of the feeding tube hub C. In example embodiments illustrated in FIGS. 3-5, the plug 10 as described herein comprises the plug member 12 or insert, a body 20 from which the plug member 12 extends, and one or more openings 30 or passageways, channels or access openings (described further below) for permitting the cleaning tool 80 to access the annular space A of the feeding tube hub C to clean the same. In example embodiments, the plug 10 can further comprise a grasping portion 40 or handle (e.g., for grasping to install/remove the plug 10 to/from the feeding tube hub C), the tether 50, and the retaining ring 55, for example, for generally tethering the plug 10 to the feeding tube hub C. In use, the tether 50 can be grasped and function as a handle to rotate the plug 10 with respect to the feeding tube hub C.

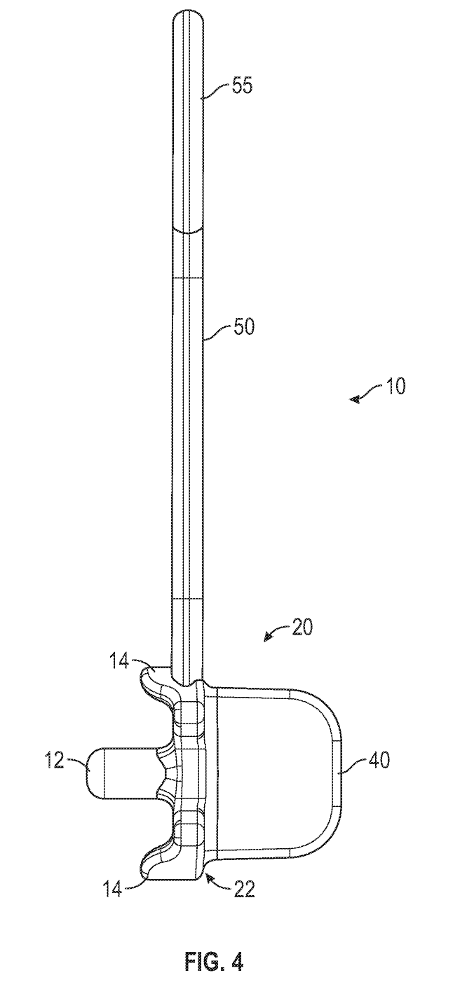

The body 20 can have a generally planar geometry with a circumferential edge 22 interrupted by the geometry of the openings 30. The body 20 can have a collar or skirt 14 that can extend generally downwardly, while being interrupted by the openings 30 or cutouts as shown particularly in FIGS. 3-4, leaving gaps in the collar for additional insertion of the brush 80. The plug 10 is capable of being attached and removed from the hub C while the cleaning tool is occupying the annular space A.

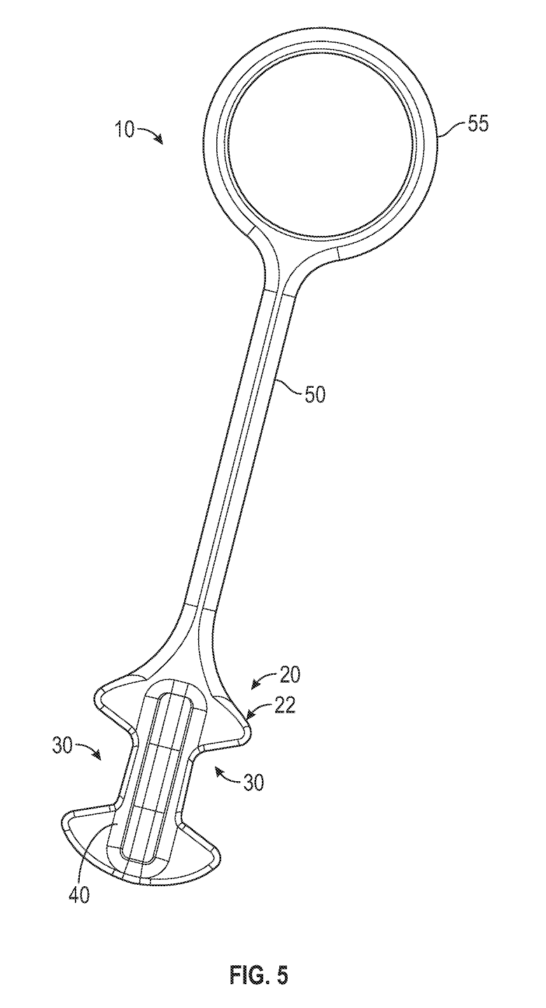

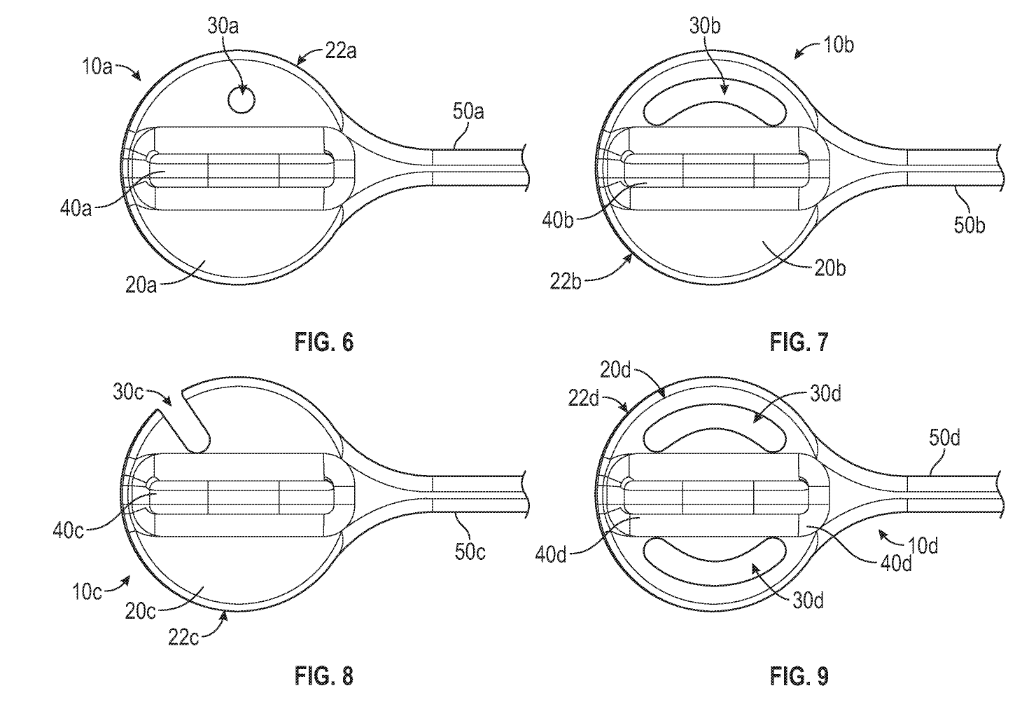

FIGS. 6-13 illustrate example designs of plugs which can effectively function as the plug (10) described in FIGS. 1-5. The plugs illustrated in FIGS. 6-13 can include similar geometries to the plug (10) in FIGS. 1-2, including having the plug member 12 for insertion within the lumen L of the connector M of the feeding tube hub C. FIG. 6 illustrates a plug 10a with a single cylindrical opening 30a or aperture extending through a portion of a body 20a or base, for example, generally centered on one side of a grasping portion 40a or handle. The body 20a can be a planar disc having a generally circumferential periphery 22a and be connected by a tether 50a. As described above, in use the brush end 82 of the cleaning tool 80 is inserted through the opening 30a to clean the annular space A of the connector C.

FIG. 7 illustrates a plug 10b design with a similar body 20b, circumferential periphery 22b, grasping portion 40b and tether 50b. The plug 10b includes an opening 30b that defines a curved or arcuate path that is formed through a portion of the body 20b along one side of the grasping portion 40b. The opening 30b can have an elongated semi-circular geometry and generally follow the curvilinear shape of the outer circumference 22b. As described above, in use the brush end 82 of the cleaning tool 80 is inserted through the opening 30b to clean the annular space A of the connector C. The elongated shape of the opening 30b allows for the orientation and position of the brush end 82 to be changed during use.

FIG. 8 illustrates a plug 10c design with a similar body 20c, circumferential periphery 22c, grasping portion 40c and tether 50c. The plug 10c includes an opening 30c defining a generally elongate channel from a portion of the body to an outer periphery portion 22c thereof. As described above, in use the brush end 82 of the cleaning tool 80 is inserted through the opening 30c to clean the annular space A of the connector C. As shown, a portion of the channel 30c that is defined near the grasping portion 40c is generally rounded, for example and according to some example embodiments to provide for seating against an outer surface of the grasping portion 84 of the cleaning tool 80.

FIG. 9 illustrates a plug 10d design with a similar body 20d, circumferential periphery 22d, grasping portion 40d and tether 50d. The plug 10d includes a pair of opposingly oriented openings 30d that define a curved or arcuate path that is formed through a portion of the body 20d along both sides of the grasping portion 40d. The openings 30d can have an elongated semi-circular geometry and generally follow the curvilinear shape of the outer circumference 22c. As described above, in use the brush end 82 of the cleaning tool 80 is inserted through the openings 30d to clean the annular space A of the connector C. The elongated shape of the openings 30d allow for the orientation and position of the brush end 82 to be changed during use.

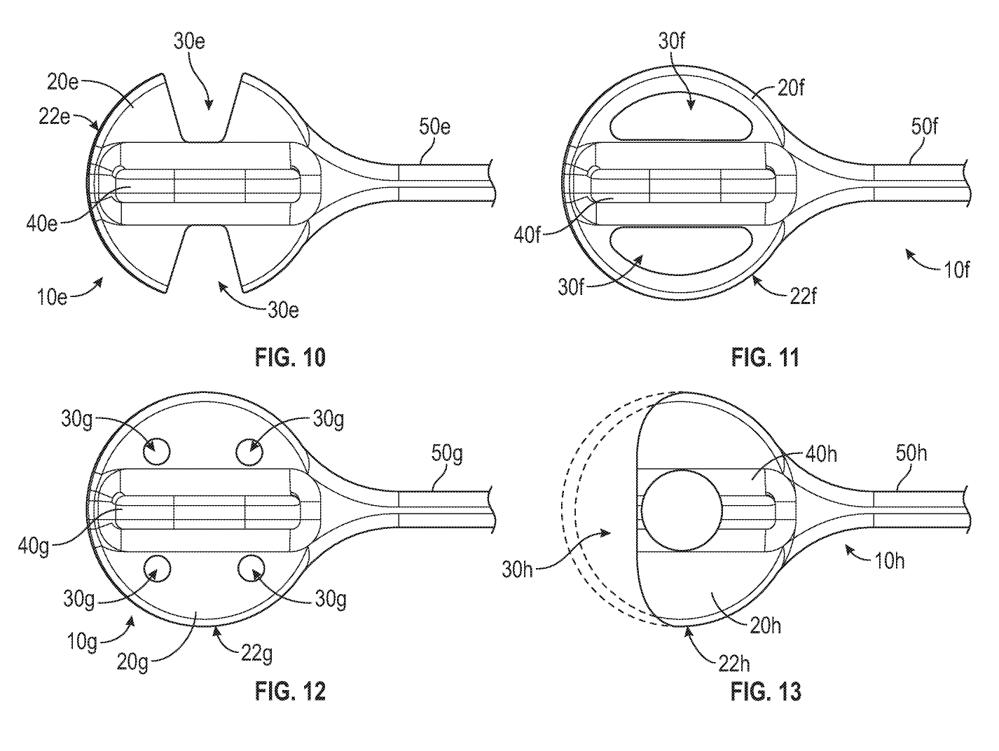

FIG. 10 illustrates a plug 10e design with a similar body 20e, circumferential periphery 22e, grasping portion 40e and tether 50e. The plug 10e includes a pair of opposingly oriented openings 30e in the form of notches or generally rectangular or polygonal openings formed through the body 20e and mirrored about an axis aligned with the grasping portion 40e. As described above, in use the brush end 82 of the cleaning tool 80 is inserted through the openings 30e to clean the annular space A of the connector C. The shape of the openings 30e allow for the orientation and position of the brush end 82 to be changed during use.

FIG. 11 illustrates a plug 10f design with a similar body 20f, circumferential periphery 22f, grasping portion 40f and tether 50f. The plug 10f includes a pair of opposingly oriented openings 30f that define a geometry that is formed through a portion of the body 20f along both sides of the grasping portion 40f. FIG. 8 is generally similar to the plug (10d) of FIG. 9, however, the interior radiused sidewall of the openings 30f or channels is removed to be substantially aligned with the extension of the grasping portion 40f, and thus the openings 30f are larger in size to provide greater flexibility in orientation and movement of the brush ends 82 of the cleaning tool 80 during use.

FIG. 12 illustrates a plug 10g design with a similar body 20g, circumferential periphery 22g, grasping portion 40g and tether 50g. FIG. 12 show a plurality of positionally separated openings 30g, for example four cylindrical openings or apertures extending through a portion of the body 20g, which can be shaped and positioned as desired to provide a variety of access positions for the brush end 82 of the cleaning tool 80. In example embodiments, the openings 30g are shaped to be substantially cylindrical in shape, however, other embodiments can comprise other shaped openings as desired, for example, oval, elliptical, rectangular, polygonal, or other desired shapes. According to one example embodiment, one or more spaced-apart openings 30g can be formed through the body 20g as desired, for example, to define a linear array of openings. According to one example embodiment, the one or more openings are substantially aligned with the annular space A.

FIG. 13 illustrates a plug 10h design with a similar body 20h, circumferential periphery 22h, grasping portion 40h and tether 50h. The plug 10h includes an opening 10h that is an entire front portion removed, for example, to define a large cutout portion from the base 22h.

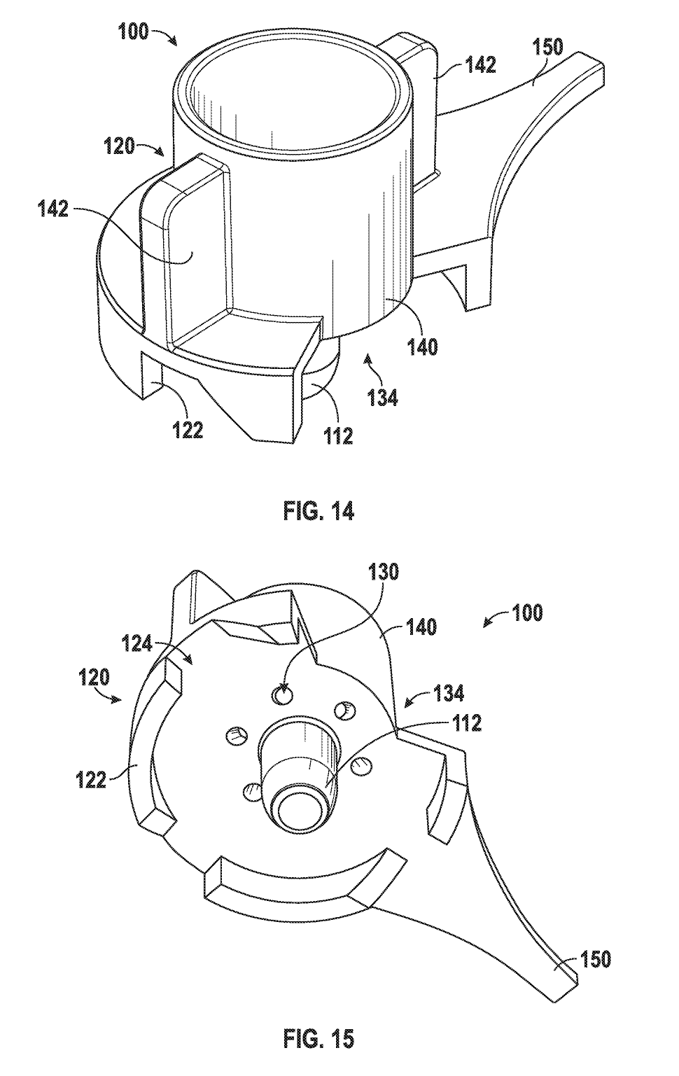

FIGS. 14-15 show a plug 100 according to another example embodiment of the present invention. In example embodiments, the plug 100 is generally similar to the plug 10 as described above, for example, comprising a plug member 112, a body 120, a collar 122 or skirt having one or more cutouts 124 defined therethrough, one or more passageways or access openings 134 for permitting the brush member 82 to access the annular space A of the feeding tube connector C when the plug 112 is sealingly engaged with the lumen L. The plug 100 further includes a syringe tip coupling 140 or collar configured for receiving engagement with a syringe tip of a syringe. The plug 100 can also have a gripping portion 142 or handle to operate similarly to the gripping portion (40) described above. The illustrated gripping portion 142 can comprise a pair of co-planar flanges extending from the syringe tip coupling 140.

In example embodiments, one or more openings, jets or channels 130 extend through the body 120 to permit cleaning fluid that is introduced within the syringe tip coupling 140 to be discharged or jetted within the annular space A of the feeding tube connector C. The channels 130 are spaced apart from each outer and generally surround the plug member 112 to define a circular array of about six generally cylindrical openings 130. The openings 130 are positioned to each fit within the open receiving area of the syringe tip coupling 140, so that fluid received into the syringe tip coupling flows uninterruptedly through the openings. In alternate embodiments, more or less than six of the openings can be provided as desired.

According to one example embodiment, the channels 130 are positioned and oriented to direct the cleaning fluid outwards (e.g., generally away from the plug member 112) and in either of a clockwise or counter-clockwise direction away from the connection axis L₁. In example embodiments, the channels 130 are configured to assist the cleaning fluid in flowing not only through the annular space A, but also around the annular space A and within the threaded portion of the collar of the connector C. For example, the channels 130 preferably provide a jetting system for cleaning the feeding tube connector C such that the cleaning fluid effectively spirals or generally moves along a helical path with respect to the connection axis L₁ as it's passed through the annular space A. In alternate example embodiments, the one or more channels 130 can be sized, positioned and oriented as desired, for example, so as to maximize the effectiveness of the cleaning fluid in removing and cleaning out any unwanted debris, bacteria, or other undesirables within the annular space A. As also described above, the brush member 82 can be introduced within the cutout 134 to occupy at least a portion of the annular space A to clean the same.

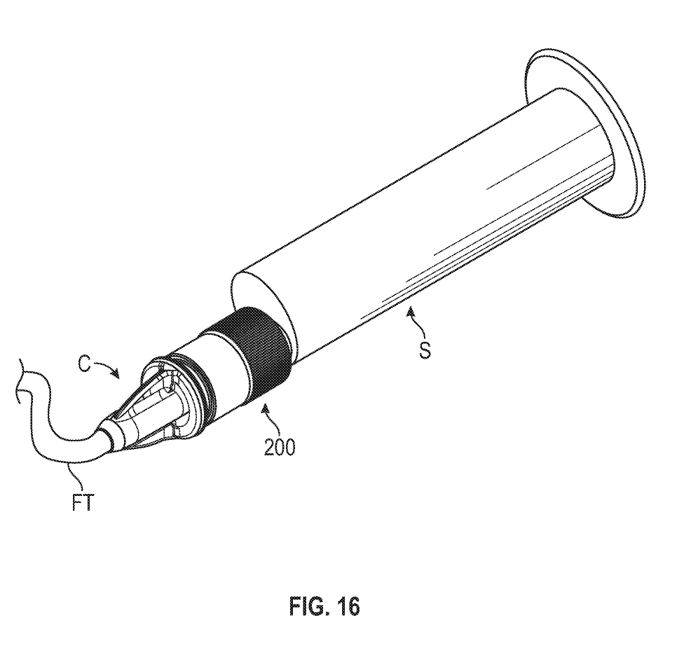

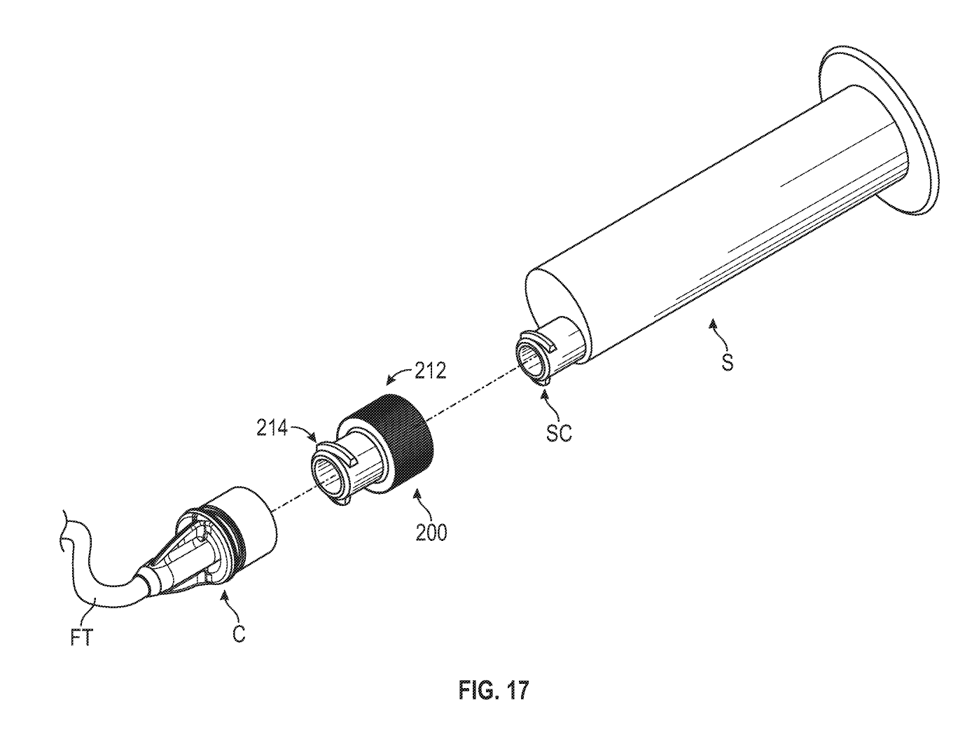

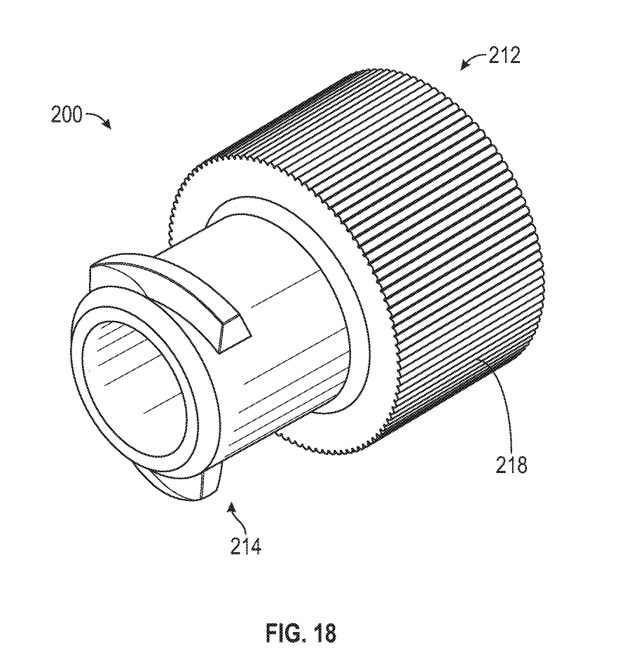

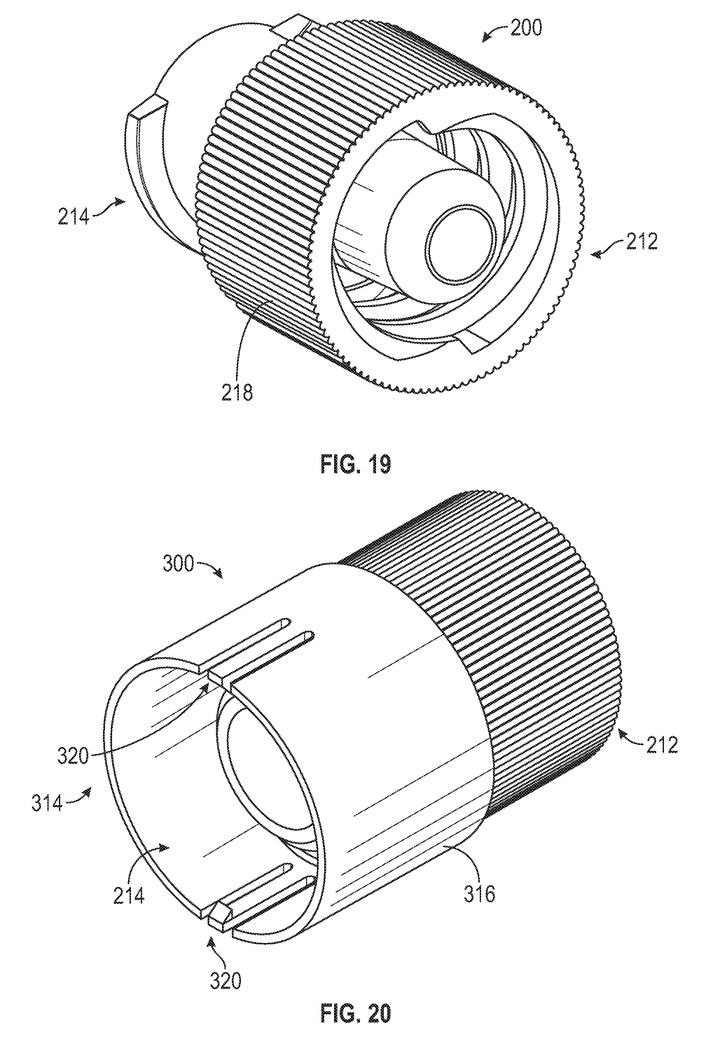

According to another example embodiment, the present invention relates to a male to female coupling or hub connector. As depicted, FIGS. 16-20 show a male to female coupling or hub connector 200 according to an example embodiment of the present invention. In example embodiments, the hub connector 200 comprises a first end 212 and a second end 214, wherein the first end 212 comprises a male ISO 80369-3 ENFit compatible or formatted coupling and the second end 214 comprises a female ISO 80369-3 ENFit compatible or formatted coupling. In example embodiments, the male to female coupling 200 is coupled between the above-described feeding tube hub or connector C and a syringe S with a female ISO 80369-3 ENFit compatible coupling SC. For example, the male ISO 80369-3 EN Fit formatted coupling on the first end 212 of the coupling 200 is configured for compatible engagement with the female coupling SC of the syringe S and the female ISO 80369-3 ENFit formatted coupling on the second end 214 of the coupling is configured for compatible engagement with a male ISO 80369-3 ENFit formatted coupling of the feeding tube connector C. In example embodiments, the feeding tube connector C can be vented. U.S. patent application Ser. No. 14/844,956 is incorporated herein by reference and discloses a vented male ISO 80369-3 EN Fit enteral coupling or connector having a housing structure with drainage passages or vents to eliminate or reduce the likelihood of retaining feeding liquids or other contaminants in the outer housing.

In example embodiments, rather than using a cleaning tool or other cleaning method for eliminating any small quantities of unused feeding fluids that might allow for bacteria colonization or contain other potential contaminants (e.g., within recesses or areas that could retain feeding fluids), the male to female coupling 200 preferably provides for a disposable solution, or for example, a solution for substantially eliminating the likelihood that unused feeding fluids would be likely to overflow within one or more portions of the feeding tube connector. For example, the male to female coupling 200 provides an intermediate conduit between the syringe female ISO 80369-3 ENFit formatted coupling and the male ISO 80369-3 ENFit formatted coupling of the feeding tube connector C. Thus, after a desirable quantity of feeding fluids or other fluids have been dispensed from the syringe S, through the coupling 200, and through the feeding tube connector C (and further through the feeding tube FT to be delivered to the patient), the components thereof can be disconnected in a way whereby substantially all of the feeding fluid, if not the entirety of the feeding fluid, is prevented from overflowing or entering within one or more recesses or areas of the feeding tube connector C, for example, such that the feeding tube connector (or portions thereof) need not be cleaned after use. For example, rather than using a cleaning device such as U.S. patent application Ser. No. 15/009,073 (the entirety thereof incorporated herein by reference), the male to female coupling 200 can be provided as an intermediate coupling between the syringe S and the feeding tube connector C.

According to one example embodiment, after feeding when it is desired to disconnect the syringe S from the feeding tube connector C, and with the male to female coupling 200 connected therebetween, the second end 214 of the male to female coupling 200 is disconnected from the male ISO 80369-3 ENFit formatted coupling of the feeding tube connector C (with the male ISO 80369-3 ENFit formatted coupling of the first end 212 remaining engaged with the female ISO 80369-3 ENFit formatted coupling of the syringe S). Preferably, the unused feeding fluids remain within the conduit of the male to female coupling 200 such that feeding fluids are prevented from overflowing or exiting from the conduit of the male ISO 80369-3 ENFit formatted coupling of the feeding tube connector C and within one or more recesses or areas of the feeding tube connector C. According to another example embodiment, with the male to female coupling 200 connected therebetween, the first end 212 of the male to female coupling 200 is disconnected from the female ISO 80369-3 ENFit formatted coupling of the syringe S (with the female ISO 80369-3 ENFit formatted coupling of the second end 214 remaining engaged with the male ISO 80369-3 ENFit formatted coupling of the feeding tube connector C). The second end 214 of the male to female coupling 200 can then be disconnected from the feeding tube connector C.

In example embodiments, the conduit extending therebetween the syringe S and the feeding tube FT can comprise a vacuum or generally become pressurized, for example, such that the initial disconnection between two of the three components (syringe, coupler 200, connector C) generally relieve the vacuum or pressure present, and wherein relieving the vacuum or pressure generally causes at least some unused or residual feeding fluids to overflow or generally become exposed to the atmosphere, some of which can enter into one or more recesses or areas of the components. Thus, example embodiments of the present invention preferably provide for redirecting or repositioning the location whereby unused feeding fluids are exposed or overflow, thereby preventing any unused feeding fluids from overflowing within one or more recesses or areas of the feeding tube connector C, and thus maintaining a clean and aseptic connector C such that a cleaning tool is not needed.

An outer collar portion of first end 212 the coupling 100 can comprise one or more surface features 218 or other recesses, indentions, projections or other features such that an enhanced gripping surface can be provided to assist a user in attachment/detachment of the coupling 200 from the connector C or syringe S. According to one example embodiment, the outer collar portion comprises a disc-like flange (not shown) or other outwardly-protruding member, for example, to ensure the coupling 200 is not a choking hazard and, for example, to comply with 16 CFR § 1501.4.

According to another example embodiment illustrated in FIG. 20, a male to female coupling 300 can be provided with a receiving collar 316 that has one or more delectably resilient fingers or clips 320 or anchors provided for secondary engagement with the feeding tube connector C. As illustrated, the clips 320 extend between a fixed end and a deflecting free anchor end. The male to female coupling 300 includes the same structure as the male to female coupling 200 illustrated above, including the identical male ISO 80369-3 ENFit formatted coupling of the first end 212 and the female ISO 80369-3 ENFit formatted coupling of the second end 214. As illustrated, the receiving collar 316 extends about and around the female ISO 80369-3 ENFit formatted coupling of the second end 214. The receiving collar 316 can have an internal diameter that is greater than the outer diameter of the outer collar OC (see FIG. 2) of the connector C in order to receive the outer collar OC therein. The pair of oppositely opposed clips 320 (comprising end protrusions extending inwardly) are provided at the second end 314 of the coupling 300, for example, such that when the female ISO 80369-3 ENFit formatted coupling of the second end 214 is fully engaged with the male ISO 80369-3 ENFit formatted coupling of the feeding tube connector C, the clips 320 generally releasably engage or anchor to an outer portion of the feeding tube connector C, for example, wherein the protrusions or projections engage with a grooved surface GS (see FIG. 2) formed along an outer body portion of the feeding tube connector C. Thus, in example embodiments, the coupling 300 preferably provides for a dual engagement or locking feature wherein a first engagement is provided between lugs of the female ISO 80369-3 ENFit coupling of the coupling 300 and the threaded collar of the feeding tube connector C, and a second engagement is provided between the clips 320 and an outer portion of the feeding tube connector C. Optionally, other clips or other complementary coupling features can be provided as desired, for example, such that an additional engagement can be provided therebetween. In some example embodiments, the clips 320 can be configured for removable engagement wherein a user can press or actuate portions thereof to release the clips from engagement with the feeding tube connector C.

Optionally, the clips 320 can be configured to permanently engage with the feeding tube connector C. According to another example embodiment, the clips or other engagement features can be provided such that axial displacement along the connection axis L₁ between the coupling 300 and the feeding tube connector C causes the clips 320 or other coupling features to disengage with the feeding tube connector. In some example embodiments, the outer sleeve or collar 316 of the coupling 300 can comprise a channel for complementary engagement with one or more protrusions formed along an outer periphery portion of the feeding tube connector C.

According to some example embodiments, the male to female coupling is configured such that engagement of a female coupling with the male coupling of the male to female coupling causes permanent engagement therebetween. For example, according to example embodiments, the male to female coupler comprises a mechanism which can cause permanent engagement with compatible couplings during its first engagement therewith. Thus, according to some example embodiments, the mechanism aids in preventing syringe re-use and/or extension set re-use.

According to another example embodiment, the syringe S and/or coupling 200 can comprise a lumen extension tip, or for example, a low-dose tip comprising a small diameter lumen, for example, such that the overflow of unused feeding fluids is further reduced (if not entirely eliminated) and wherein the volume of the conduit is reduced to a volume that is substantially similar to the volume of the conduit when the syringe is connected directly to the feeding tube connector. For example, the syringe S can comprise a lumen extension tip SLDT and the coupling 200 can comprise a lumen extension tip, both of which are configured to occupy at least a portion of the conduit of the male ISO 80369-3 ENFit formatted couplings, thereby reducing the volume of the conduit. U.S. patent application Ser. No. 15/210,282 filed Jul. 14, 2016 and U.S. patent application Ser. No. 15/659,323 disclose syringes and couplings/connectors comprising lumen extension tips, the entirety of which is incorporated by reference herein in its entirety.

According to another example embodiment, the male to female coupling 200 can also comprise a tethered plug such as plug (10) in FIGS. 1-5 coupled thereto, for example, to comply with 16 CFR § 1501.4 (e.g., not entirely fitting within the test cylinder specified by § 1501.4) and to provide for plugging the lumen or conduit of the male ISO 80369-3 ENFit formatted coupling of the feeding tube connector C.

Optionally, according to some example embodiments, one or more of the clips (320) can provide for removable or permanent engagement with the female ISO 80369-3 ENFit formatted coupling of the syringe S, for example, wherein the outer threaded collar of the second end 214 of the couplings described herein can be modified to provide for dual-action attachment and removal with the female ISO 80369-3 ENFit formatted coupling of the syringe. Optionally, the outer collar of the feeding tube connector C can similarly comprise a modified or clipped collar with a similar structure to the collar 316 and clips 320 herein described. U.S. patent application Ser. No. 15/454,761, U.S. patent application Ser. No. 15/078,674, U.S. patent application Ser. No. 15/185,583, U.S. patent application Ser. No. 14/844,922, U.S. Design Patent Application Serial No. 29/521,665, and U.S. Design Patent Application Serial No. 29/533,173 are incorporated herein by reference and disclose various clipped, snap-on and dual-action attachment and removal mechanisms for replacement with the outer coaxial connection collars of the male to female couplings 200, 300 and/or the feeding tube connector C.

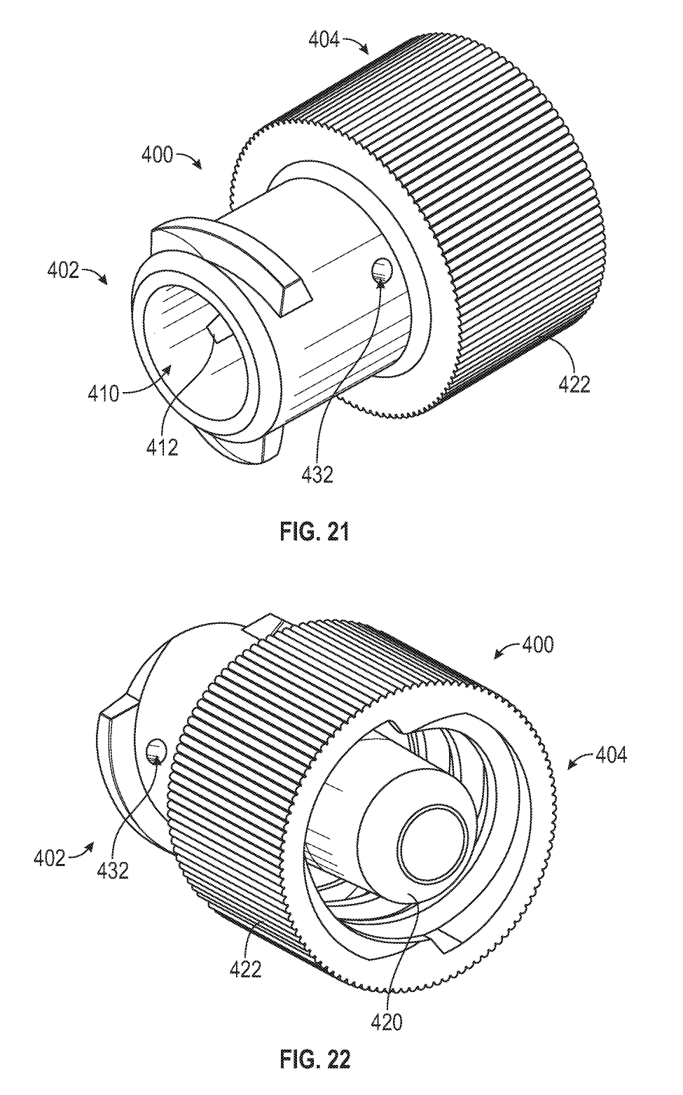

FIGS. 21-24 illustrates a plug 400 for plugging the lumen L of a feeding tube connector C (see FIGS. 1-2) according to additional example embodiments of the present invention. For example, the plug 400 can be generally tethered to the feeding tube connector C, similarly to the embodiment shown in FIGS. 1-2, and connectable to the coupling M of the feeding tube connector C while sealing the lumen L thereof and permitting a syringe S or other device containing a cleaning fluid to connect and output the cleaning fluid through one or more openings such that the cleaning fluid passes through the annular space A of the connector C to clean or flush the same. In example embodiments, the plug 400 comprises a first coupling end 402 for coupling engagement with the feeding tube connector C and a second coupling end 404 for coupling engagement with a syringe tip ST of the syringe S. According to one example embodiment, the syringe S comprises an oral syringe having a standard oral syringe tip.

In example embodiments, the first coupling end 402 comprises an ISO 80369-3 formatted coupling for providing for coupling engagement with the connector M of the feeding tube connector C. In example embodiments, the first coupling end 402 defines a reservoir 410 that is to be occupied by the connector M, and a plug member 412 is generally centrally-positioned therein for providing sealing engagement with the lumen L of the connector M. One or more channels, jets or openings 430 are defined generally near the plug member 412 and communicate with the second coupling end 404. In example embodiments, the second coupling end 404 comprises an inner syringe coupling 420 (e.g., for coupling engagement with the syringe tip ST) and an outer collar portion 422 generally surrounding the syringe coupling 420. In example embodiments, the one or more openings 430 are configured to communicate between the syringe coupling 420 and the reservoir of the first coupling end 402. An opening or conduit 432 is provided on a portion of the first coupling end 602. The one or more openings 430 can be fluidly connected to the conduit(s) 432 through a circular array of six channels formed through the body, each of the channels extending generally outwards away from the central plug member and in one of either the clockwise or counter-clockwise directions. The cleaning fluid passing through the channels spirals or generally moves along a helical path as it's passed through the annular space from the openings 430 to the exit from the conduit(s) 432.

In example embodiments, the conduit 432 provides a path such that the cleaning fluid passing through the one or more openings 430 is guided or directed to flow within the annular space A and along the one or more threads defined along an internal portion of the collar of the feeding tube connector C. For example, with the connector M generally occupying most of the reservoir 410 of the first coupling end 402 and with the plug member 412 sealingly engaging the lumen L of the connector M, any cleaning fluid introduced within the syringe coupling 420 flows through the one or more openings 430 and within the reservoir 410. However, as the connector M is occupying a substantial amount of the reservoir 410 (and generally sealingly engaged therewith), the cleaning fluid therein is prevented from flowing out of the reservoir 410, but instead takes the path of least resistance and further flows through the conduits 432 and within the annular space A and along the threads of the collar of the feeding tube connector C. According to one example embodiment, one conduit 432 is formed through first coupling end 402. In alternate example embodiments, two or more conduits can be formed through the first coupling end 402.

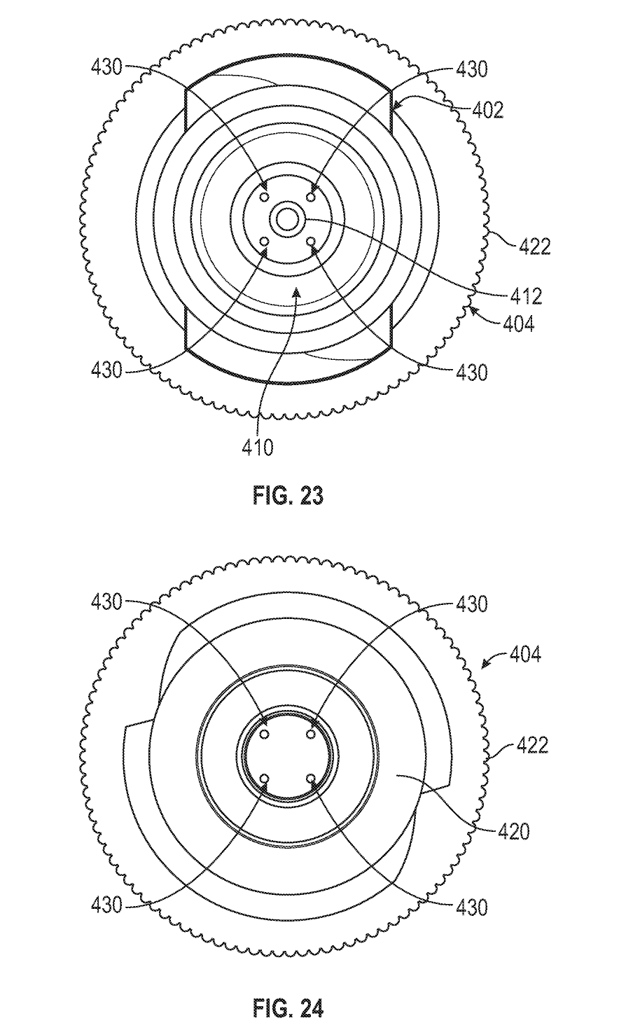

An additional embodiment can include elements of the plug structure similar to the plug (10) (described in FIGS. 1-5) and elements of the coupling (200, 400) structure in FIGS. 16-19, 23-24. Such a combination of these above-described embodiments can be determined for enablement without explicit illustration. Similarly to the above-described plug (10), this additional plug comprises a plug member, a body from which the plug member extends, and a tether extending from the body. Also, a skirt or outer flange can be formed along an outer periphery portion of the body, for example, which generally abut an end portion of the connector C when the plug is sealingly engaged with the lumen L of the connector M. Additionally, a side of the body that is generally opposite the side comprising the plug member comprises a syringe tip coupling similar to the first end (212) of the coupling (200) described in FIGS. 16-19, and one or more wings or gripping portions generally extending outwardly from the syringe tip coupling. As similarly described above, one or more openings, similar to openings (430) in FIG. 23-24, are formed through the body to provide a path for cleaning fluid to flow through the plug and within the annular space A of the connector C to clean the same. The opening can be positioned and formed through the body such that cleaning fluid introduced in the syringe tip coupling flows through the body and exits at a position spaced away from the plug member to further flow within the annular space A and along the threads of the collar of the feeding tube connector C. Similarly to the plug (10) described above, one or more reduced surface area void sections or cutouts can be defined within portions of the body such that a brush or cleaning tool 80 can pass therethrough to occupy one or more portions of the annular space to clean the same.

In example embodiments, the body can include a skirt extending about at least a portion of the periphery thereof. In example embodiments, at least a portion of the skirt comprises one or more reduced surface area passageways or cutouts. In example embodiments, the body comprises a single cutout for permitting the brush 80 (and brush end 82) access within the annular space A. As similarly described above, while the brush member 82 is at least partially occupying the annular space A and with the plug member sealed with the lumen L, the plug can be rotated in either of the clockwise or counter-clockwise directions such that the brush member 82 can access and clean the entirety of the surfaces defining the annular space A.

According to example embodiments, some of the plugs as described herein can provide both access within the annular space A and the introduction of a cleaning fluid within the annular space, both of which can be accomplished while the lumen L of the connector M remains sealed.

While the invention has been described with reference to example embodiments, it will be understood by those skilled in the art that a variety of modifications, additions and deletions are within the scope of the invention, as defined by the following claims.

What is claimed is:

1. A plug for plugging a feeding tube hub comprising a male coupling defining a lumen extending therethrough, an outer collar and an annular space defined therebetween, the plug comprising:
a base configured for releasably covering at least a portion of an annular space defined in an outer collar of a feeding tube hub;
a plug insert extending from the base, the plug insert being configured for plugging a lumen of a feeding tube hub along a connection axis; and
wherein at least one passageway extends through the base to provide access to the annular space defined in the outer collar of the feeding tube hub when the plug insert is plugging the lumen of the feeding tube hub.

2. The plug of claim 1, wherein the base is rotatable about the connection axis with respect to the feeding tube hub annular space when the plug insert is plugging the feeding tube hub lumen.

3. The plug of claim 2, further comprising a handle extending from the base, the handle being configured to manipulate rotation of the base about the connection axis with respect to the feeding tube hub annular space.

4. The plug of claim 3, wherein the handle comprises a tether rotatably secured to the feeding tube hub, the tether configured to flexibly form a gripping geometry when the plug insert is plugging the feeding tube hub lumen.

5. The plug of claim 3, wherein the handle comprises a flange oriented along a planar axis that is configured to not cover the at least one passageway extending through the base.

6. The plug of claim 1, wherein the at least one base passageway is configured to provide for insertion of a cleaning tool into the feeding tube hub annular space when the plug insert is plugging the feeding tube hub lumen.

7. The plug of claim 6, wherein the at least one base passageway comprises an aperture extending through the base.

8. The plug of claim 6, wherein the at least one base passageway comprises a cutout from the base.

9. The plug of claim 1, wherein the base comprises a skirt oriented about the plug insert, the skirt being configured to releasably engage the feeding tube hub outer collar when the plug insert is plugging the feeding tube hub lumen.

10. The plug of claim 9, wherein at least one passageway extending through the base comprises at least one reduced surface area in the skirt configured to provide an opening between the skirt and the feeding tube hub outer collar when the plug insert is plugging the feeding tube hub lumen.

11. The plug of claim 1, further comprising a fluid conduit extending from the base in a direction away from the plug insert, the fluid conduit being configured to receive a volume of fluid from a fluid source and deliver the volume of fluid through the at least one passageway in the base to the annular space in the outer collar of the feeding tube hub when the plug insert is plugging the feeding tube lumen.

12. The plug of claim 11, wherein the fluid conduit comprises a collar oriented about the connection axis.

13. The plug of claim 11, wherein the fluid conduit comprises a syringe tip connector.

14. The plug of claim 11, further comprising a fluid exit positioned external to the plug insert and the at least one passageway with respect to the connection axis, the fluid exit configured to release the volume received within the annular space in the outer collar of the feeding tube hub when the plug insert is plugging the feeding tube lumen.

15. The plug of claim 14, wherein the fluid exit comprises at least one reduced surface area in a skirt extending from the base and oriented about the plug insert.

16. A coupler for cleaning a feeding tube hub comprising a male coupling defining a lumen extending therethrough, an outer collar and an annular space defined therebetween, the coupler comprising:
a base configured for releasably covering at least a portion of an annular space defined in an outer collar of a feeding tube hub;
a seal configured for removably sealing a lumen of a feeding tube hub; and
a fluid conduit configured for receiving a volume of fluid from a fluid source and delivering the volume of fluid to the annular space in the outer collar of the feeding tube hub when the seal is sealing the feeding tube lumen.

17. The coupler of claim 16, wherein the fluid conduit comprises a syringe tip connector.

18. The coupler of claim 16, further comprising a fluid exit configured to release the volume received within the annular space in the outer collar of the feeding tube hub when the seal is sealing the lumen of the feeding tube hub.

19. The coupler of claim 18, wherein the fluid exit comprises at least one reduced surface area in a skirt extending from the base and oriented about the seal.

20. The coupler of claim 16, wherein the fluid conduit comprises at least one aperture extending through the base.

21. The coupler of claim 16, wherein the base comprises: a male ISO 80369-3 formatted coupling for receiving the volume of fluid from the fluid source, and a female ISO 80369-3 formatted coupling for coupling to the feeding tube hub.

22. A coupling system comprising:
a fluid delivery female ISO 80369-3 formatted coupling;
a hub male ISO 80369-3 formatted coupling being separate from the fluid delivery female ISO-80369-3 formatted coupling; and
a coupler comprising a female ISO 80369-3 formatted coupling and a male ISO 80369-3 formatted coupling, the coupler being separate from the fluid delivery female ISO 80369-3 formatted coupling and the hub male ISO 80369-3 formatted coupling, the coupler female ISO 80369-3 formatted coupling being removably coupled to the hub male ISO 80369-3 formatted coupling, and the coupler male ISO 80369-3 formatted coupling being removably coupled to the fluid delivery female ISO 80369-3 formatted coupling.

23. The coupling system of claim 22, wherein the coupler comprises a conduit extending therethrough, the coupler conduit is configured to fluidly connect the fluid delivery ISO 80369-3 formatted coupling and the hub male ISO 80369-3 formatted coupling.

24. The coupling system of claim 23, wherein the fluid delivery female ISO 80369-3 formatted coupling is part of a syringe, and the hub male ISO 80369-3 formatted coupling is part of a feeding tube.

25. The coupling system of claim 24, further comprising at least one anchor to be releasably anchored with respect to the feeding tube.

26. The coupling system of claim 25, wherein the at least one anchor comprises a clip.

27. The coupling system of claim 26, wherein the clip comprises a resiliently deflectable arm extending between a fixed end and a clip end.

28. The coupling system of claim 25, further comprising a collar oriented about the coupler female ISO 80369-3 formatted coupling, the collar being configured to receive the hub male ISO 80369-3 formatted coupling therein.

29. A method of cleaning a feeding tube hub comprising:
providing a feeding tube hub, the feeding tube hub being in communication with a lumen of a feeding tube and comprising a male coupling defining a lumen extending therethrough, an outer collar and an annular space defined therebetween;
providing a cleaning tool;
providing a feeding tube hub plug, the feeding tube hub plug comprising a plug member for sealing engagement with the lumen of the male coupling, a body extending from the plug member, and at least one opening or cutout defined in the body;
attaching the plug member with the feeding tube hub to seal the lumen of the male coupling;
inserting at least a portion of the cleaning tool within the at least one opening or cutout of the feeding tube plug hub such that at least a portion of the brush end is present within the annular space of the feeding tube hub;
rotating the feeding tube hub plug relative to the feeding tube hub while the cleaning tool remains at least partially present within the annular space; and
removing the cleaning tool from the at least one opening or cutout of the feeding tube hub plug.

30. The method of claim 29, further comprising comprises applying a cleaning fluid to the cleaning tool prior to insertion of the cleaning tool within the annular space.

31. The method of claim 29, further comprising discharging at least some cleaning fluid directly within the annular space prior to inserting the cleaning tool within the annular space.

32. A plug for a feeding tube connector, the feeding tube hub comprising a male coupling defining a lumen extending therethrough, an outer collar and an annular space defined therebetween, the feeding tube hub plug comprising:
a body;
a central plug member extending from a first side of the body for plugging a lumen of a feeding tube hub;
a syringe tip coupling extending from a second side of the body, the syringe tip coupling configured for compatible engagement with a syringe tip of a syringe; and
one or more channels extending through the body such that a cleaning fluid introduced within the syringe tip coupling flows through the body and within the annular space to clean the same.

33. The plug of claim 32, comprising a circular array of six channels formed through the body, each of the channels extending generally outwards away from the central plug member and in one of either the clockwise or counter-clockwise directions.

34. The plug member of claim 33, wherein the cleaning fluid passing through the channels spirals or generally moves along a helical path as it's passed through the annular space.

35. The plug member of claim 32, wherein at least one opening or cutout is defined in the body such the annular space is accessible without unplugging the central plug member from the lumen of the feeding tube hub.
